(19) United States
(12) Patent Application Publication
(10) Pub. No.: US 2025/0041580 A1
(43) Pub. Date: Feb. 6, 2025

(54) KINK-REDUCING IV LINES AND EXTENSION SETS

(71) Applicant: The Nemours Foundation, Jacksonville, FL (US)

(72) Inventor: Phoebe FISHER, Coatesville, PA (US)

(21) Appl. No.: 18/792,224

(22) Filed: Aug. 1, 2024

Related U.S. Application Data
(60) Provisional application No. 63/530,564, filed on Aug. 3, 2023.

(51) Int. Cl. A61M 39/08

(52) U.S. Cl. CPC A61M 39/08; A61M 2205/02

(57) ABSTRACT

A kink-resistant medical tubing including a tubular member defining a lumen therethrough, the tubular member including a first end, a second end, and a middle portion positioned therebetween, the first end operable to couple to secondary member and a kink-resistant member positioned along the tubular member proximate the first end, the kink-resistant member operable to limit bending of the tubular member such that a bend of the tubular member has a predefined minimum bending radius, wherein the kink-resistant member includes a first spacer and a second spacer operable to contact the first spacer to define the predefined minimum bending radius.

### RELATED APPLICATION

This application is related to and claims priority from U.S. Provisional Application No. 63/530,564, filed Aug. 3, 2023, the disclosure of which is incorporated herein by reference in its entirety.

### TECHNICAL FIELD

Various aspects of the present disclosure relate to intravenous (IV) lines and extension sets. In some specific examples, various aspects of the present disclosure are directed toward apparatuses, systems, and methods that reduce the likelihood of kinking and bending of IV lines and extension sets during use.

### BACKGROUND

The process of securing peripheral IV lines has evolved over time. Adhesive dressings have improved to keep a peripheral line attached to the patient without tearing skin. In pediatrics, an extension piece was added years ago, and only more recently has it been brought into practice with adults. The addition of the extension of 15 cm to 20 cm allows for the exchange of infusion tubing without needing to disturb the tape on the IV catheter and the patient. In the pediatric population, the most commonly used extension has been a T-connector with a needle port (see FIG. 1-A). This allows the extension tubing to be brought around 180° without kinking, to keep it well secured under the tape and away from the fingers of pediatric patients.

An effort has been made in recent years to go to a strictly needle-free port system. Needle-stick injuries are a major safety concern for healthcare staff. A T-connector with a needle-free port (see FIG. 1-C) has been created, but it is bulky, especially for pediatric patients. It also provides a large and noticeable object that attracts the attention of the child. This can result in moving, dislodging, and discomfort for the patient.

In some cases, straight connectors (see FIG. 1-B) are now being used on both children and adults. Straight connectors are less bulky and provide the appropriate functionalities, especially when a port is not needed in such close proximity to the patient. The problem is that the existing straight connectors kink very easily.

The kinking of IV lines is not only inconvenient but can be dangerous to patients. A kink can slow or stop the delivery of critical fluids and/or medication to a patient. When a line partially or fully kinks, the flow and velocity of flow decreases or stops. This can allow a clot to form inside the IV catheter. Once a catheter has a clot, it is generally not salvageable and needs to be replaced. If the extension is on an arterial line, that clot can become an embolus with significant consequences. Often medications and fluids need to be administered in a timely fashion. If the IV is compromised, those medications and fluids need to be held until a new IV access can be established.

An additional problem with the straight connector is that, in the effort to keep it from promptly kinking, a large loop must be created when it is secured in the usual 180 degree fashion. This loop is easier for children to grab and pull out the IV.

When infusion tubing is connected, there is a pull on the extension by the tubing. Even the most well-placed and taped extension will kink given time, due to the pull of the infusion tubing. When a connector kinks, the tape on the IV and kinked extension must be taken down and the extension unkinked and resecured. IVs can easily be lost in this process. Kinked extensions cause IV alarms to go off throughout the day and night on the floor. The alarms and the need for re-doing the tape requires time and attention and increases the stress for patients and healthcare staff. There is a high risk of losing an IV in this process, requiring another IV to be placed.

In the operating room, kinking can happen under the drapes and be inaccessible.

Furthermore, because of the smoothness of the tubing, tape used to secure the tubing is unable to securely adhere to the tubing. This allows the extension tubing to slide under the tape, increasing the likelihood of kinking.

This problem exists in the straight extension used in pediatrics as well as the larger bore straight extensions used in adults.

FIGS. 1-3 illustrate various existing medical systems that are implemented that are either susceptible to kinking (see FIG. 3) or that are large and difficult to use in certain populations such as pediatric patients (see FIG. 2).

Various aspects of the present disclosure are directed toward overcoming these shortcomings, as well as other additional or alternative advantages.

### SUMMARY

Various aspects of the present disclosure are directed toward apparatuses, systems, and methods that include a kink-reducing IV lines and extension sets.

In an embodiment a kink-resistant medical tubing includes a tubular member defining a lumen therethrough, the tubular member including a first end, a second end, and a middle portion positioned therebetween, the first end operable to couple to secondary member; and a kink-resistant member positioned along the tubular member proximate the first end, the kink-resistant member operable to limit bending of the tubular member such that a bend of the tubular member has a predefined minimum bending radius, wherein the kink-resistant member includes a first spacer and a second spacer operable to contact the first spacer to define the predefined minimum bending radius.

In an embodiment further to the previous embodiment, the kink-resistant member includes a plurality of spacers including the first spacer and the second spacer, wherein each of the plurality of spacers are operable to contact adjacent spacers of the plurality of spacers.

In an embodiment further to the previous embodiments, the plurality of spacers includes beading.

In an embodiment further to the previous embodiments, the beading is integrally formed on the tubular member.

In an embodiment further to the previous embodiments, the beading is coupled to the tubular member.

In an embodiment further to the previous embodiments, each bead of the beading has a diameter of about 0.6 cm.

In an embodiment further to the previous embodiments, each bead extends from an outer diameter of the tubular member about 0.2 cm.

In an embodiment further to the previous embodiments, each bead of the beading has a sloped side, wherein the sloped side is angled at about 60 degrees relative to an outer surface of the tubular member.

In an embodiment further to the previous embodiments, the beading includes six beads, wherein the six beads are operable to facilitate a maximum of a 180 degree turn in the tubular member along a longitudinal position covered by the six beads.

In an embodiment further to the previous embodiments, each bead is about 0.8 cm long along a length of the tubular member.

In an embodiment further to the previous embodiments, the predefined minimum bending radius is about 0.8 cm.

In an embodiment further to the previous embodiments, the kink resistant member includes slip-resistant members positioned longitudinally spaced from the first and second spacers along the length of the tubular member.

In an embodiment, a kink-resistant member for resisting kinking of a tubular member, the kink-resistant member configured to be coupled to the tubular member, the kink-resistant member includes a plurality of beads operable to be coupled to a tubular member, each bead of the plurality of beads including a sloped side, wherein adjacent sloped sides of each of the plurality of beads define an angle therebetween, wherein the angle is about 60 degrees.

In an embodiment further to the previous embodiments, each bead of the plurality of beads has a diameter of about 0.6 cm.

In an embodiment further to the previous embodiments, the plurality of beads is positioned on a sleeve, the sleeve configured to be positioned about the tubular member.

In an embodiment further to the previous embodiments, the sleeve includes a longitudinal slit configured to facilitate placement on the tubular member.

In an embodiment further to the previous embodiments, the plurality of beads are integral with the sleeve.

In an embodiment further to the previous embodiments, the sleeve includes an adhesive operable to adhere.

In an embodiment further to the previous embodiments, each bead of the plurality of beads is configured to extend from an outer diameter of the tubular member about 0.2 cm.

In an embodiment further to the previous embodiments, the plurality of beads includes six beads to facilitate a maximum of a 180 degree turn in a longitudinal line extending through each of the six beads. the tubular member along a longitudinal position covered by the six beads.

While multiple, inventive examples are specifically disclosed, various modifications and combinations of features from those examples will become apparent to those skilled in the art from the following detailed description. Accordingly, the disclosed examples are meant to be regarded as illustrative in nature and not restrictive.

### BRIEF DESCRIPTION OF THE DRAWINGS

FIG. 1 illustrates various traditional connectors for IV lines;

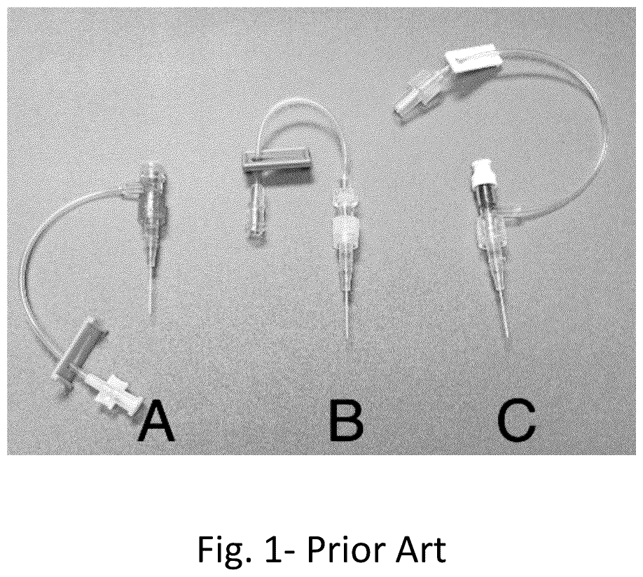

FIG. 2 illustrates an IV setup in which a T-connector with a needle port in a hand positions the IV line outside of the profile of the hand;

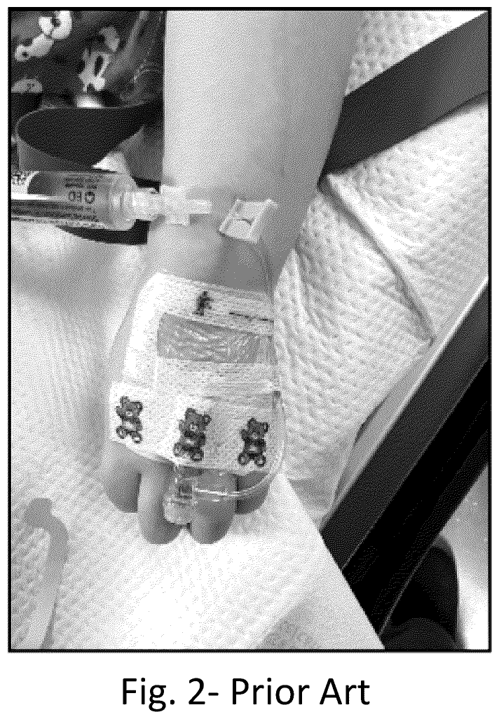

FIG. 3 illustrates an IV setup in which a straight connector is positioned in the hand in which the IV line is kinked;

FIGS. 4A and 4B illustrate a kink-resistant device, in accordance with an embodiment;

FIGS. 5A and 5B illustrate a kink-resistant device including spacers configured to resist kinking or collapse of an internal lumen of a tubular member, in accordance with an embodiment;

FIGS. 6A and 6B illustrate the kink-resistant device of FIGS. 9A and 9B in the curved configuration in which the lumen is maintained in an unimpeded configuration, in accordance with an embodiment; and

FIG. 7 illustrates a kink-resistant member with a sleeve operable to be positioned on a tubular member, in accordance with an embodiment.

### DETAILED DESCRIPTION

Various aspects of the present disclosure are directed toward apparatuses, systems, and methods that include a device for resisting kinking of lines. In various examples, the device, as discussed herein, helps provide support to lines that experience bends and are thus prone to kinking, leading to compromised flow through the lumen of the line. In certain instances, the components of the device can be integral to a device, such as in the IV extension example, or can exist as a stand-alone piece that can be added to an existing system.

A kink-resistant device 10 is provided herein to reduce the likelihood of kinking of a line during use. Although discussed in specific use-cases herein, it is understood that the lines discussed herein may be implemented in various settings, scenarios, and even industries. For example, as discussed herein, one example of a kink-resistant device 10 includes an IV fluid delivery line that may be used with infant and pediatric populations, which present unique challenges to fluid delivery due to anatomy, scale, and self-control. It is understood that similar arrangements may be used for adult and geriatric populations, for cardiac lines, Foley catheters, and so forth. This may be implemented in various setting including fluid and medication delivery. This may be especially beneficial in settings in which coagulation or aggregation within the line may be a risk. For example, certain settings may have a longer procedure time and may include risk of clotting or other clogs in the line. The systems and methods presented herein may include dialysis, chemotherapy, delivery of particles in suspensions, delivery of blood, and so forth. Reduction of kinking in lines results in less coagulation of blood and less aggregation of particles during procedures implementing IV lines. Additionally, such lines that resist kinking may be useful in other industries that implement lines that require or are susceptible to bending along specific lengths of lines, especially with respect to attachment points such as nozzles and the like.

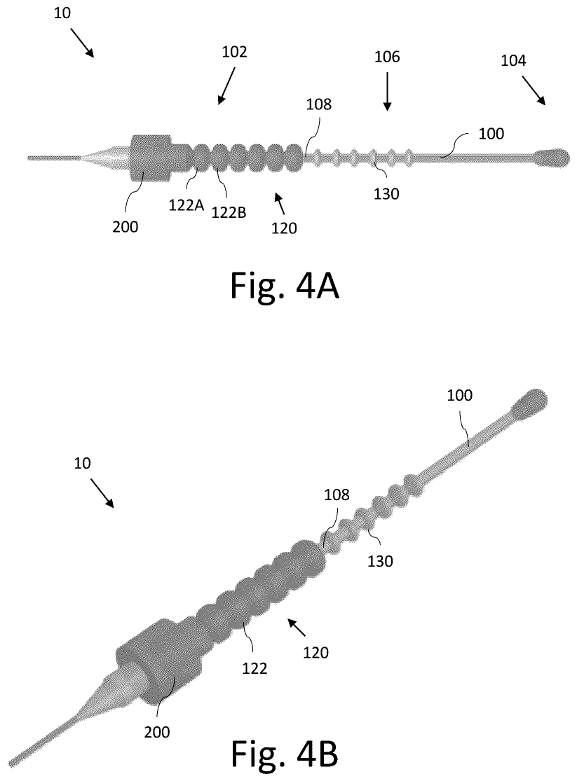

Referring specifically to one use-case, but not to be limited to such, FIG. 4 depicts a kink-resistant device 10. The kink-resistant device 10 may be implemented with various connectors such as Luer locks, fittings, and so forth. As outlined in the background, lines may be susceptible to kinking, and may be especially susceptible near fittings and connectors. This is due to the rigid nature of fittings and connectors. The kink-resistant device 10 is implemented to limit kinking at positions near such connectors or at positions otherwise with a greater likelihood of kinking.

Some advantages of the kink-resistant device 10 include limiting kinking of lines. This may be achieved by including a device with a kink-resistant portion or securing a device over a line where it needs to bend. The device then guides the line through the bend in a controlled fashion over a limited distance, without substantially increasing stiffness of the line. This may also be achieved without increasing the profile of the line to a profile greater than the fitting to which it is coupled. This may also be achieved without substantially disrupting the visual field through the line in those lines that are at least semi-transparent. This may also be achieved in such a way that the line maintains 360-degree bendability without rotation of the line as secured (e.g., via medical adhesives to a patient). This may also be achieved with the ability to bend in multiple directions.

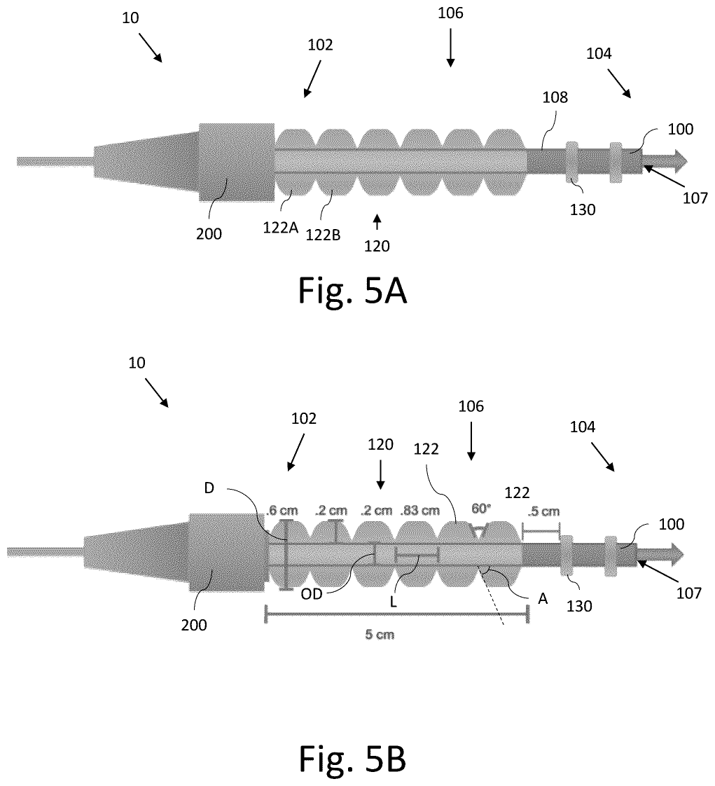

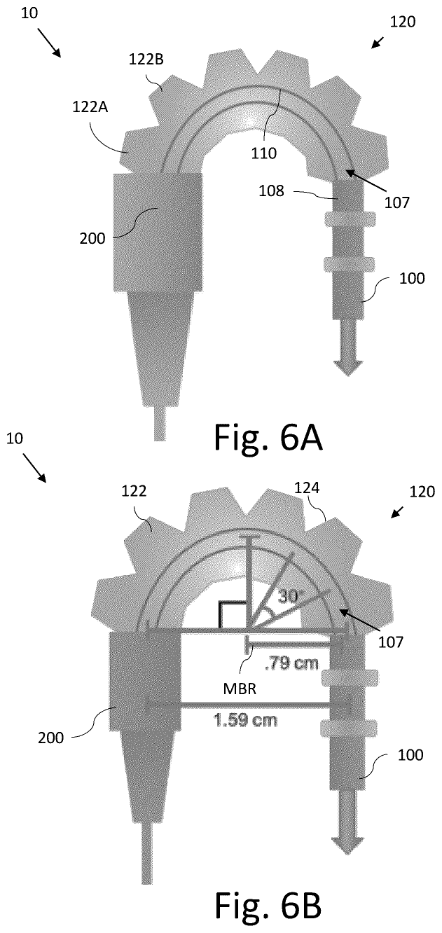

The kink-resistant device 10 illustrated in FIGS. 4A and 4B is a tubular member 100 (e.g., a IV line, a catheter, and so forth) and includes a first end 102 and a second end 104. The tubular member 100 includes a middle portion 106 extending between the first end 102 and the second end 104. A lumen 107 is defined through the tubular member 100 (see FIGS. 5A-6B). The tubular member 100 includes an outer surface 108 and an inner surface 110, wherein the inner surface 110 defines the lumen 107. The outer surface 108 may not be the outer-most surface of the tubular member 100, but instead may represent generally the outer profile of the tubular member 100 at portions not including additional structures or protrusions that are discussed herein. It is understood that various configurations of the tubular member 100 may be implemented with various lumen diameters, wall thicknesses, materials, and so forth.

The first end 102 of the tubular member 100 is coupled to a secondary structure 200 such as a Luer lock or fitting. In order to resist kinking at or near the secondary structure, the tubular member 100 is provided with a kink-resistant member 120. The kink resistant member 120 is operable to reinforce the tubular member 100 at a specified position to limit bending and turning of the tubular member 100 beyond a predetermined geometry.

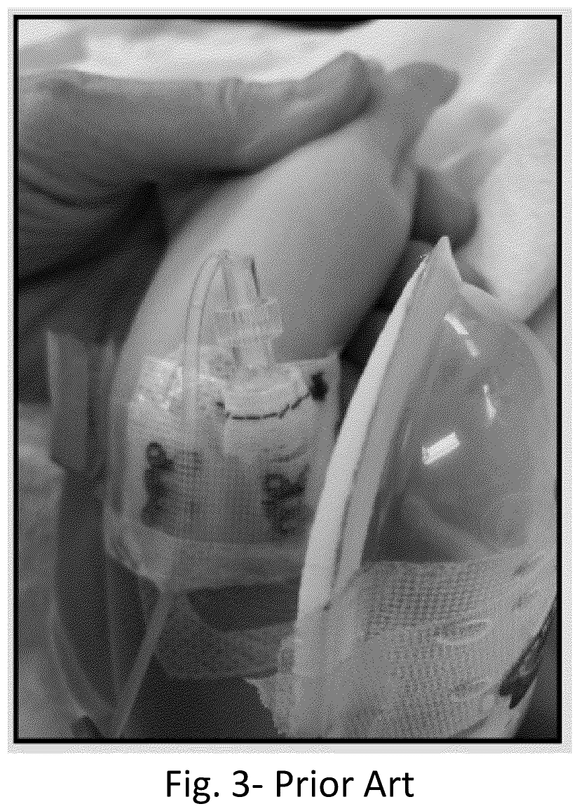

In some embodiments, the kink-resistant member 120 is positioned along the tubular member 100 proximate the first end 102. The kink-resistant member 120 may be positioned proximate the first end 102 as the first end is a common position at which tubing kinks as is illustrated in FIG. 3 when not supported be a kink-resistant member. It is understood that the kink-resistant member 120 may be positioned at other locations and is still within the scope of this disclosure. The kink-resistant member 120 is operable to limit bending of the tubular member 100 such that a bend of the tubular member has a predefined minimum bending radius. This is accomplished by providing a plurality of spacers 122. For example, the kink-resistant member 120 includes a first spacer 122A and a second spacer 122B. The spacers 122 are configured to contact each other to limit the amount of bending the tubular member 100 experiences at a longitudinal position at which the spacers 122 are positioned. This allows the spacers to define the predefined minimum bending radius of the tubular member 100 at the kink-resistant member 120. The first spacer 122A may also be positioned flush against a secondary structure 200 to specifically limit kinking at the position at which the tubular structure 100 couples to the secondary structure 200.

In some embodiments, the spacers 122 are positioned along the longitudinal length of the tubular member 100. The spacers 122 may be contacting each other in a neutral position (e.g., unbent position) or they may be slightly spaced from each other. However, as the tubular member 100 is bent, adjacent spacers come into contact or greater contact along a surface of the spacers 122 to provide mechanical interference against each other to limit further bending of the tubular member 100. The geometry and spacing of the spacers 122 may be tailored to provide various diameters, bending profiles, and so forth.

In one embodiment, the spacers 122 may be represented as a bead or beading. Bead is to be understood broadly to include spacers which include a lumen therethrough. The bead may be integral with the kink-resistant member 120, the tubular member 100, or other spacers, or may be individually or collectively coupled to the kink-resistant member 120, the tubular member 100, or other structures. The bead may be formed of the same material or different material of the kink-resistant member 120, the tubular member 100, or other structures. The bead for example, may be integrally formed with the tubular member 100 and thus is formed of the same material as the tubular member 100 (e.g., PVC with plasticizers, polyethylene, polypropylene, etc.). It is understood that the bead may formed of the same, similar or different materials and by different material processes and techniques are still within the scope of the disclosure.

Although a specific example is provided herein as to the size, diameter, and geometry of the kink-resistant member 120, it is understood that other sizes, diameters, and geometries may be implemented and are still within the scope and spirit of this disclosure. Additionally, ranges are provided herein for some of the features. However, it is understood that the devices of this disclosure are applicable in various settings and industries and thus may be at different scales. Accordingly, this disclosure is not meant to be strictly limiting to the ranges and sizes provided herein, but may be seen as examples.

Referring to FIGS. 5A and 5B, in some embodiments, each spacer 122 or bead may have a diameter D of about 0.6 cm. It is understood that the spacers 122 may have greater or lesser diameters as applied on different tubular member 100 sizes and types. For example, the spacers may have a diameter from about 0.1 cm to about 0.2 cm, from about 0.2 cm to about 0.3 cm, from about 0.3 cm to about 0.4 cm, from about 0.4 cm to about 0.5 cm, from about 0.5 cm to about 0.6 cm, from about 0.6 cm to about 0.7 cm, from about 0.7 cm to about 0.8 cm, from about 0.8 cm to about 0.9 cm, from about 0.9 cm to about 1.0 cm, from about 1.0 cm to about 1.5 cm, from about 1.5 cm to about 2.0 cm, from about 2.0 cm to about 3.0 cm, and greater. The spacers 122 may protrude from or beyond the outer surface 108 of the tubular member 100. Thus, although the spacers 122 may have a specific diameter D, the spacers 122 may only protrude from the surface of the tubular member 100 a shorter distance. For example, the spacers 122 may protrude from the outer surface 108 of the tubular member 100 from about 0.1 cm to about 0.2 cm, from about 0.2 cm to about 0.3 cm, from about 0.3 cm to about 0.4 cm, from about 0.4 cm to about 0.5 cm, from about 0.5 cm to about 0.6 cm, from about 0.6 cm to about 0.7 cm, from about 0.7 cm to about 0.8 cm, from about 0.8 cm to about 0.9 cm, from about 0.9 cm to about 1.0 cm, from about 1.0 cm to about 1.5 cm, from about 1.5 cm to about 2.0 cm, from about 2.0 cm to about 3.0 cm, and greater. In some embodiment, the diameter D of the spacer 122 may be a ratio of the diameter of the tubular member 100. For example the diameter D of the spacer 122 relative to the diameter of the tubular member 100 (e.g., diameter to the outer surface 108 of the tubular member 100) is from about 1.5:1 to about 1.6:1, from about 1.6:1 to about 1.7:1, from about 1.7:1 to about 1.8:1, from about 1.8:1 to about 1.9:1, from about 1.9:1 to about 2.0:1, from about 2.0:1 to about 2.1:1, from about 2.1:1 to about 2.2:1, from about 2.2:1 to about 2.3:1, from about 2.3:1 to about 2.4:1, from about 2.4:1 to about 2.5:1, from about 2.5:1 to about 2.6:1, from about 2.6:1 to about 2.7:1, from about 2.7:1 to about 2.8:1, from about 2.8:1 to about 2.9:1, from about 2.9:1 to about 3.0:1, from about 3.0:1 to about 3.1:1, from about 3.1:1 to about 3.2:1, from about 3.2:1 to about 3.3:1, from about 3.3:1 to about 3.4:1, from about 3.4:1 to about 3.5:1, from about 3.5:1 to about 3.6:1, from about 3.6:1 to about 3.7:1, from about 3.7:1 to about 3.8:1, from about 3.8:1 to about 3.9:1, from about 3.9:1 to about 4.0:1, from about 4.0:1 to about 4.5:1, and from about 4.5:1 to about 5.0:1.

In some embodiments, the diameter D of the spacers 122 are less than the outer diameter OD of the secondary structure 200. This prevents the tubular member 100 and the kink-resistant member 120 from having a larger profile than the secondary structure 200. This can be helpful when placing an IV as a kink-resistant member 120 having a diameter greater than the secondary structure 200 would increase the puncture angle or result in ramping of the IV line during use.

In some embodiments, each spacer 122 or bead may include a longitudinal length L of about 0.83 cm. It is understood that the spacers 122 may have greater or lesser longitudinal lengths as applied on different tubular member 100 sizes and types. For example, the spacers may have a diameter from about 0.1 cm to about 0.2 cm, from about 0.2 cm to about 0.3 cm, from about 0.3 cm to about 0.4 cm, from about 0.4 cm to about 0.5 cm, from about 0.5 cm to about 0.6 cm, from about 0.6 cm to about 0.7 cm, from about 0.7 cm to about 0.8 cm, from about 0.8 cm to about 0.9 cm, from about 0.9 cm to about 1.0 cm, from about 1.0 cm to about 1.5 cm, from about 1.5 cm to about 2.0 cm, from about 2.0 cm to about 3.0 cm, from about 3.0 cm to about 4.0 cm, from about 4.0 cm to about 5.0 cm, and greater. In some embodiment, the longitudinal length L of the spacer 122 may be a ratio of the diameter of the tubular member 100. For example the longitudinal length L of the spacer 122 relative to the diameter of the tubular member 100 (e.g., diameter to the outer surface 108 of the tubular member 100) is from about 1.0:1 to about 1.1:1, from 1.1:1 to about 1.2:1, from 1.2:1 to about 1.3:1, from 1.3:1 to about 1.4:1, from 1.4:1 to about 1.5:1, from 1.5:1 to about 1.6:1, from about 1.6:1 to about 1.7:1, from about 1.7:1 to about 1.8:1, from about 1.8:1 to about 1.9:1, from about 1.9:1 to about 2.0:1, from about 2.0:1 to about 2.1:1, from about 2.1:1 to about 2.2:1, from about 2.2:1 to about 2.3:1, from about 2.3:1 to about 2.4:1, from about 2.4:1 to about 2.5:1, from about 2.5:1 to about 2.6:1, from about 2.6:1 to about 2.7:1, from about 2.7:1 to about 2.8:1, from about 2.8:1 to about 2.9:1, from about 2.9:1 to about 3.0:1, from about 3.0:1 to about 3.1:1, from about 3.1:1 to about 3.2:1, from about 3.2:1 to about 3.3:1, from about 3.3:1 to about 3.4:1, from about 3.4:1 to about 3.5:1, from about 3.5:1 to about 3.6:1, from about 3.6:1 to about 3.7:1, from about 3.7:1 to about 3.8:1, from about 3.8:1 to about 3.9:1, from about 3.9:1 to about 4.0:1, from about 4.0:1 to about 4.5:1, and from about 4.5:1 to about 5.0:1

In some embodiments, the spacers 122 may be provided with rounded edges to limit chafing, scratching, or digging into skin of the patient during use.

Although the spacers 122 are described as having a diameter, the actual shape can vary. Thus, the diameter D mentioned herein should be understood to represent a width of a portion of the spacer 122.

Referring to FIGS. 6A and 6B, the geometry of each spacer 122 or bead is provided to permit a minimum bend radius MBR. For example, each spacer 122 may include a side 124 that is sloped. The side 124 may be a flat or curved but the effective slope of the side is based on the point of contact between adjacent spacers 122. In one embodiment, the side 124 of the spacer 122 is angled to define an angle A at about 60 degrees relative to the outer surface 108 of the tubular member 100. This permits the tubular structure 100 to have a 180 degree turn with the use of six spacers 122 or beads at the portion of the tubular structure 100 including the kink-resistant member 120 (e.g., each spacer 122 or bead represents about a 30 degree turn of the tubular member 100, see FIG. 6B). It is understood that any number of angles A may be implemented for the side 124 to achieve the appropriate MBR when implemented in combination with the length L of the spacer 122 (e.g., angel A could be from about 45 degrees to about 50 degrees, from about 50 degrees to about 55 degrees, from about 55 degrees to about 60 degrees, from about 60 degrees to about 65 degrees, from about 65 degrees to about 70 degrees, from about 70 degrees to about 75 degrees, from about 75 degrees to about 80 degrees, and from about 80 degrees to about 85 degrees). When each spacer 122 has a longitudinal length L of about 0.83 cm and a side 124 that is angled at about 60 degrees relative to the outer surface 108 of the tubular member 100, this allows the tubular member to have a minimum bend radius of about 0.79 cm. The geometry of the spacers 122 may be modified to provide the appropriate or desired minimum bend radius MBR and degrees of turning.

Different minimum bend radii MBR and degrees of turning may be advantageous in various settings. For example, a smaller minimum bend radius MBR may be advantageous for an IV line on a pediatric patient such that the IV line may be turned 180 degrees while maintaining the IV line within the profile of the patient's anatomy (e.g., the hand), which limits overhang off of the anatomy which in turn limits unintended snags on objects or accessibility of the IV line to the pediatric patient. In other settings, a larger minimum bending radius MBR may be desirable, such as a larger IV line that might potentially experience kinking with such a tight minimum bending radius MBR. Thus, one of skill in the art is capable of tuning the geometry of the spacers 122 to achieve the appropriate minimum bend radius MBR and the desired maximum degree of turning 180. In some embodiments, the geometry of the spacers 122 may be provided to allow for a minimum bend radius MBR of from about 0.4 cm to about 0.5 cm, from about 0.5 cm to about 0.6 cm, from about 0.6 cm to about 0.7 cm, from about 0.7 cm to about 0.8 cm, from about 0.8 cm to about 0.9 cm, from about 0.9 cm to about 1.0 cm, from about 1.0 cm to about 1.5 cm, from about 1.5 cm to about 2.0 cm, from about 2.0 cm to about 3.0 cm, from about 3.0 cm to about 4.0 cm, from about 4.0 cm to about 5.0 cm, and greater. This may results in various ratios of the size of the spacer 122 (i.e., diameter of the spacer 122) to the size of the line (i.e., outer diameter of the line), including but not limited to from about 5:1 to about 4:1, from about 4:1 to about 3:1, from about 3:1 to about 2:1, and from about 2:1 to about 1.5:1. Additionally the ratio of the diameter of the line to the diameter of the lumen of the line in combination with the size of the spacer 122 may be optimized to limit kinking such that the size of the spacer 122 may be optimized for reduction of kinking and reduction of profile.

In some embodiments, the geometry of the spacers 122 and the number of spacers 122 may be provided to achieve a maximum degree of turning from about 30 degrees to about 45 degrees, from about 45 degrees to about 55 degrees, from about 55 degrees to about 65 degrees, from about 65 degrees to about 75 degrees, from about 75 degrees to about 90 degrees, from about 90 degrees to about 105 degrees, from about 105 degrees to about 115 degrees, from about 115 degrees to about 125 degrees, from about 125 degrees to about 135 degrees, from about 135 degrees to about 145 degrees, from about 145 degrees to about 155 degrees, from about 155 degrees to about 165 degrees, from about 165 degrees to about 180 degrees, from about 180 degrees to about 200 degrees, from about 200 degrees to about 220 degrees, from about 220 degrees to about 240 degrees, from about 240 degrees to about 260 degrees, from about 260 degrees to about 280 degrees, from about 280 degrees to about 300 degrees, from about 300 degrees to about 320 degrees, from about 320 degrees to about 340 degrees, from about 340 degrees to about 360 degrees, and greater.

Because the spacers 122 surround the tubular member 100, the tubular member 100 has a minimum bending radius MBR can be in any direction 360 degrees about the tubular member 100. This allows the tubular member 100 to be bent in any direction or have multiple bends in different directions. This avoids the need to be aware of the clocking of the tubular member 100 during use. Stated otherwise, the bending of the tubular member 100 at the kink-resistant member 120 is not constrained to a specific plane.

In some embodiments, the tubular member 100 and/or the kink-resistant member 120 may include slip-resistant members 130 positioned longitudinally spaced from the spacers 122 along the length of the tubular member 100. The slip-resistant members 130 extend beyond or protrude from the outer surface 108 of the tubular member 100. The slip-resistant members 130 are positioned longitudinally spaced from the spacers 122 and from each other. The slip-resistant members 130 provide structure to which tapes and adhesives may grip during use. As discussed herein, IV lines are often taped down to a patient to secure the line and the insertion point. However, because of various forces that are applied to IV lines (e.g., internal and external forces) during movement, infusion, and so forth, and because of the smooth outer surface of IV lines, the IV lines are susceptible to sliding beneath the medical taping, which can cause kinking, movement or dislodging of the insertion point, and so forth. This can be both painful and dangerous for the patient. The slip-resistant members 130 provides additional structure around which medical taping can be applied. The slip-resistant members 130 may be provided along any length of the tubular member 100. In one embodiment, the slip-resistant members 130 are provided along the tubular member 100 from about 0.5 to about 0.75 cm intervals for about 5 cm after the spacers 122. It is understood that the slip-resistant members 130 may be provided at any number of intervals and for any lengths along the tubular member 100. The additional profile of the slip-resistant members 130 allows for the medical taping (not shown) to grip around the slip-resistant members 130 and be adhered to the skin of a patient in such a way that slipping through the medical taping is limited. This is because the medical tape is able to adhere to the skin of the patient in a pattern that creates pockets around the slip-resistant members 130. The pockets limit movement of the slip-resistant members 130 outside each of the pocket.

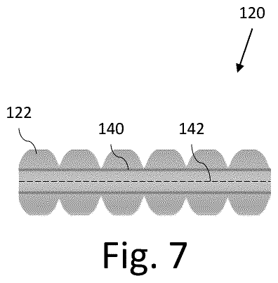

As discussed, the kink-resistant member 120 may be integral with or separate from the tubular member 100. Referring to FIG. 7, in some embodiments, when the kink-resistant member 120 is separate from the tubular member 100, the kink-resistant member 120 may be provided as an attachment. In some embodiments, each spacer 122 may be coupled individually to the tubular member 100. In other embodiments, the kink-resistant member 120 may include the spacers 122. The kink-resistant member 120 may include a sleeve 140 which is capable of being coupled to the tubular member 100. The sleeve 140 may be formed of various materials, including the same material as the tubular member 100 or other materials. For example, in one embodiment, the sleeve 140 may be formed of a thin layer of material (e.g., a film) from which the spacers 122 extend. The sleeve 140 may include an adhesive that facilitates coupling between the kink-resistant member 120 and the tubular member 100. The sleeve 140 may also be bonded directly to the tubular member 100 (e.g., heat or chemical bonding).

In some embodiments, the sleeve 140 may be threaded onto the tubular member 100. In other embodiments, the sleeve 140 may include a slit 142 such that the sleeve 140 may be installed onto the tubular member 100 without access to one of the first end 102 or the second end 104. The slit 142 extends longitudinally along the sleeve. The spacers 122 or beads may be provided as described above. In some embodiments, the sleeve 142 may also include the slip-resistant members 130 as described herein.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The particular implementations shown and described herein are illustrative examples of the invention and are not intended to otherwise limit the scope of the invention in any way. The use of any and all examples, or exemplary language (e.g., “such as”) provided herein, is intended merely to better illuminate the invention and does not pose a limitation on the scope of the invention unless otherwise claimed. Numerous modifications and adaptations will be readily apparent to those skilled in this art without departing from the spirit and scope of the invention.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening.

The recitation of ranges of values herein are merely intended to serve as a shorthand method of referring individually to each separate value falling within the range, unless otherwise indicated herein, and each separate value is incorporated into the specification as if it were individually recited herein.

Terms such as “about” or “approximately”, unless otherwise defined or restricted in the specification, should be understood to define a variance of plus or minus 5%-10% to the numerical term referred to.

All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not impose a limitation on the scope of the invention unless otherwise claimed. The various embodiments and elements can be interchanged or combined in any suitable manner as necessary.

The use of directions, such as forward, rearward, top and bottom, upper and lower are with reference to the embodiments shown in the drawings and, thus, should not be taken as restrictive. Reversing or flipping the embodiments in the drawings would, of course, result in consistent reversal or flipping of the terminology.

No language in the specification should be construed as indicating any non-claimed element as essential to the practice of the invention.

It will be apparent to those skilled in the art that various modifications and variations can be made to the present invention without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalents.

What is claimed is:

1. A kink-resistant medical tubing comprising:
a tubular member defining a lumen therethrough, the tubular member including a first end, a second end, and a middle portion positioned therebetween, the first end operable to couple to secondary member; and
a kink-resistant member positioned along the tubular member proximate the first end, the kink-resistant member operable to limit bending of the tubular member such that a bend of the tubular member has a predefined minimum bending radius, wherein the kink-resistant member includes a first spacer and a second spacer operable to contact the first spacer to define the predefined minimum bending radius.

2. The kink-resistant medical tubing of claim 1, wherein the kink-resistant member includes a plurality of spacers including the first spacer and the second spacer, wherein each of the plurality of spacers are operable to contact adjacent spacers of the plurality of spacers.

3. The kink-resistant medical tubing of claim 2, wherein the plurality of spacers includes beading.

4. The kink-resistant medical tubing of claim 3, wherein the beading is integrally formed on the tubular member.

5. The kink-resistant medical tubing of claim 3, wherein the beading is coupled to the tubular member.

6. The kink-resistant medical tubing of claim 3, wherein each bead of the beading has a diameter of about 0.6 cm.

7. The kink-resistant medical tubing of claim 6, wherein each bead extends from an outer diameter of the tubular member about 0.2 cm.

8. The kink-resistant medical tubing of claim 3, wherein each bead of the beading has a sloped side, wherein the sloped side is angled at about 60 degrees relative to an outer surface of the tubular member.

9. The kink-resistant medical tubing of claim 8, wherein the beading includes six beads, wherein the six beads are operable to facilitate a maximum of a 180 degree turn in the tubular member along a longitudinal position covered by the six beads.

10. The kink-resistant medical tubing of claim 3, wherein each bead is about 0.8 cm long along a length of the tubular member.

11. The kink-resistant medical tubing of claim 1, wherein the predefined minimum bending radius is about 0.8 cm.

12. The kink-resistant medical tubing of claim 1, wherein the kink resistant member includes slip-resistant members positioned longitudinally spaced from the first and second spacers along the length of the tubular member.

13. A kink-resistant member for resisting kinking of a tubular member, the kink-resistant member configured to be coupled to the tubular member, the kink-resistant member comprising:
a plurality of beads operable to be coupled to a tubular member, each bead of the plurality of beads including a sloped side, wherein adjacent sloped sides of each of the plurality of beads define an angle therebetween, wherein the angle is about 60 degrees.

14. The kink-resistant member of claim 13, wherein each bead of the plurality of beads has a diameter of about 0.6 cm.

15. The kink-resistant member of claim 13, wherein the plurality of beads is positioned on a sleeve, the sleeve configured to be positioned about the tubular member.

16. The kink-resistant member of claim 15, wherein the sleeve includes a longitudinal slit configured to facilitate placement on the tubular member.

17. The kink-resistant member of claim 15, wherein the plurality of beads are integral with the sleeve.

18. The kink-resistant member of claim 15, wherein the sleeve includes an adhesive operable to adhere.

19. The kink-resistant member of claim 13, wherein each bead of the plurality of beads is configured to extend from an outer diameter of the tubular member about 0.2 cm.

20. The kink-resistant member of claim 13, wherein the plurality of beads includes six beads to facilitate a maximum of a 180 degree turn in a longitudinal line extending through each of the six beads. the tubular member along a longitudinal position covered by the six beads.
